(19) United States
(12) Patent Application Publication
(10) Pub. No.: US 2026/0308087 A1
(43) Pub. Date: Oct. 8, 2026

(54) SYSTEMS AND METHODS FOR USING FACILITATED TRANSPORT MEMBRANES TO SEPARATE OLEFINS AND PARAFFINS

(71) Applicant: Compact Membrane Systems, Inc., New Castle, DE (US)

(72) Inventors: Sudipto MAJUMDAR, New Castle, DE (US); Kenneth J. PENNISI, New Castle, DE (US)

(21) Appl. No.: 19/484,623

(22) PCT Filed: Jun. 4, 2024
(86) PCT No.: PCT/US2024/032438

§ 371 Date: Nov. 14, 2025

Related U.S. Application Data
(60) Provisional application No. 63/471,055, filed on Jun. 5, 2023.
(60) Provisional application No. 63/471,058, filed on Jun. 5, 2023.

(51) Int. Cl. C07C 7/00; B01D 3/14; B01D 53/22; B01D 69/14; B01D 71/32; C07C 7/04; C07C 7/144; C08F 10/06

(52) U.S. Cl. CPC C07C 7/005; B01D 3/145; B01D 53/228; B01D 53/229; B01D 69/142; B01D 71/32; C07C 7/04; C07C 7/144; C08F 10/06; B01D 2256/24; B01D 2257/702

(57) ABSTRACT

The present disclosure provides systems and methods for separating an olefin from a paraffin. The systems and methods can include enriching an olefin from a feedstock comprising an olefin and a paraffin using a distillation column. The present disclosure also provides systems and methods for producing a polyolefin which can include separating un-reacted olefin from a reactor purge stream using a facilitated transport membrane. Among other advantages, the systems and methods described herein can increase overall polyolefin production from a factory, produce a paraffin-rich product, reduce energy demand and greenhouse gas emissions associated with a splitter column, and change the energy mixture for separation toward electrical energy.

### BACKGROUND

Olefins are the building blocks of the plastics industry and are used in the production of a high percentage of consumer goods. The production of these plastics begins with the thermal cracking of straight chain hydrocarbons with single bonds, known as alkanes or paraffins to produce a hydrocarbon know as alkenes or olefins (i.e., molecules containing carbon atoms with double bonds). It is these highly reactive olefins (e.g., ethylene, propylene, butylene, butene, etc.) that are reacted with a catalyst in a polymerization reactor to form the polyolefin molecules we know as plastics.

The systems and methods described herein can address challenges related to reduced motor fuels demand over time and a shift to lighter feedstocks in the petrochemical industry (i.e., from naphtha to liquified petroleum gas (LPG)) as it relates to the production of polyolefins. These challenges, coupled with the ever-increasing consumer demand for polyolefins, can lead to feed stock storage and high price volatility in the marketplace.

### SUMMARY

Recognized herein are various technical challenges related to separating olefins and paraffins having two (C2), three (C3), and four (C4) carbon atoms. These molecules are typically separated using distillation columns (i.e., splitter columns). However, due to the close boiling points of the olefins and paraffins for a given number of carbon atoms, the process is both energy and capital intensive. The distillation columns typically have a plurality (e.g., more than 120) high-efficiency trays and operate at high reflux ratios. The number of trays in the column makes the process capital intensive and the high reflux ratio makes the process energy intensive. There is trade-off between the number of trays in the column and the reflux ratio. The more trays in the column, the lower the reflux ratio can be used to achieve the same separation. Thus, capital cost can be traded-off for energy cost.

In recent years, many existing olefin/paraffin distillation processes have become fully loaded with feed as demand for product has increased. Often, the columns contain the highest efficiency trays that are available and there is no means to obtain further capacity gains other than replace the existing column or install an additional column. However, both of these options are very capital intensive.

The present disclosure provides systems and methods that address the abovementioned shortcomings by incorporating a membrane separation module in association with an (e.g., existing) distillation column. The membrane can be a facilitated transport membrane, e.g., having an ionomer and a carrier agent. The carrier agent can be a group 11 metal (copper, silver, gold).

Additionally, the present disclosure provides systems and methods that address the abovementioned shortcomings by incorporating a membrane separation module on the purge stream from the polymerization reactor. The membrane can separate olefin from paraffin in this stream, returning an olefin-rich stream to the polymerization reactor.

The present systems and methods are advantageous in several ways. These include, for example, increasing the overall polyolefin production of the factory, increasing production of a paraffin-rich stream, reducing energy demand at the splitter column, reducing greenhouse gas emissions associated with the splitter column reboiler, and changing the energy mix for separation of the increased feed to be more weighted toward electrical energy.

Overall, the systems and methods described herein can also eliminate the need to or replace an existing column or add an additional distillation column. A lower capital cost can be achieved compared to increasing distillation capacity. Energy can be saved for this additional capacity relative to distillation.
1. In one aspect, the disclosure provides a method for separating olefin from paraffin, the method comprising:
   a. enriching an olefin from a feedstock comprising an olefin and a paraffin using a distillation column, wherein the distillation column (i) has a plurality of separation trays, (ii) produces a top product that is enriched in olefin relative to the feedstock, and (iii) produces a bottom product that is depleted in olefin relative to the feedstock; and
   b. using a facilitated transport membrane that is capable of separating olefin from paraffin, performing one or more of:
      i. separating olefin from paraffin in the feedstock to produce an enriched feedstock, which enriched feedstock is enriched in olefin relative to the feedstock, and wherein the enriched feedstock is fed to the distillation column,
      ii. separating olefin from paraffin in the top product of the distillation column,
      iii. separating olefin from paraffin in the bottom product of the distillation column, and
      iv. withdrawing a side stream from the distillation column in proximity to a first separation tray of the plurality of separation trays and separating olefin from paraffin in the side stream.

In some embodiments, at least two of (b)(i-iv) are performed. In other embodiments, at least three of (b)(i-iv) are performed. In other different embodiments, all of (b)(i-iv) are performed.

In any of the foregoing aspects, the olefin comprises ethylene, propylene, butylene, butene, or any combination thereof. In other embodiments,, wherein the paraffin comprises ethane, propane, butane, or isobutane.

In some embodiments, the feedstock is an output of a thermal cracking process that utilizes naphtha or a heavy liquid stream from a crude distillation unit. For example, in some embodiments the thermal cracking process is a steam cracking or fluidic catalytic cracking (FCC) process.

In other aspects, the method further comprises, in a reactor, polymerizing an enriched olefin stream to produce a polyolefin. For example, the enriched olefin stream may comprise between about 95 weight percent (wt%) and about 99.5 wt% olefin.

In some embodiments, the facilitated transport membrane comprises an ionomer, such as a fluorinated ionomer.

In different aspect of the method, the facilitated transport membrane comprises a carrier agent. For example, the carrier agent is a group 11 metal ion in some embodiments.

In other embodiments, the facilitated transport membrane is a hollow-fiber membrane or a spiral-wound flat sheet membrane.

In different embodiments, the facilitated transport membrane is humidified. In other embodiments, the facilitated transport membrane is associated with a module that combines continuous addition of water vapor to form a humidified input stream and selective permeation of an olefin in the humidified input stream using the facilitated transport membrane.

In yet more embodiments of the method, producing the enriched feedstock in (b)(i), further produces a concentrated paraffin stream or a concentrated olefin stream. In other embodiments, the separation in (b)(ii) produces a concentrated paraffin stream, a concentrated olefin stream, and/or a recycle stream that is returned to the distillation column. In more embodiments, the separation in (b)(iii) produces a concentrated paraffin stream, a concentrated olefin stream, and/or a recycle stream that is returned to the distillation column. In yet more embodiments, the separation in (b)(iv) produces a concentrated paraffin stream, a concentrated olefin stream, and/or a recycle stream that is returned to the distillation column. For example, in some aspects, wherein (i) a first recycle stream enriched in olefin compared to the side stream is returned to the distillation column at a location above the first separation tray and (ii) a second recycle stream enriched in paraffin relative to the side stream is returned to the distillation column at a location below the first separation tray.

Another aspect provides a system for separating olefin from paraffin, the system comprising:
c. a distillation column having a plurality of trays, which distillation column is configured to (i) enrich an olefin from a feedstock comprising an olefin and a paraffin, (ii) produce a top product that is enriched in olefin relative to the feedstock, and (iii) produces a bottom product that is depleted in olefin relative to the feedstock; and
d. facilitated transport membrane that is capable of separating olefin from paraffin and configured to perform one or more of:
   i. separate olefin from paraffin in the feedstock to produce an enriched feedstock, which enriched feedstock is enriched in olefin relative to the feedstock, and wherein the enriched feedstock is configured to be fed to the distillation column,
   ii. separate olefin from paraffin in the top product of the distillation column,
   iii. separate olefin from paraffin in the bottom product of the distillation column, and
   iv. withdraw a side stream from the distillation column in proximity to a first separation tray of the plurality of separation trays and separate olefin from paraffin in the side stream.

In some embodiment, the facilitated transport membrane is configured to perform at least two of (b)(i-iv) or the facilitated transport membrane is configured to perform at least three of (b)(i-iv) or the facilitated transport membrane is configured to perform all of (b)(i-iv).

In different embodiments of the system, the olefin comprises ethylene, propylene, butylene, butene, or any combination thereof, while in other embodiments, the paraffin comprises ethane, propane, butane, or isobutane.

In other embodiments of the system, the feedstock is an output of a thermal cracking process that utilizes naphtha or a heavy liquid stream from a crude distillation unit. For example, in some embodiments the thermal cracking process is a steam cracking or fluidic catalytic cracking (FCC) process.

In still more embodiments, the system further comprises a reactor configured to polymerize an enriched olefin stream to produce a polyolefin. For example, in some embodiments the enriched olefin stream comprises between about 95 weight percent (wt%) and about 99.5 wt%

In other embodiments of the system, the facilitated transport membrane comprises an ionomer, for example in some embodiments the ionomer is fluorinated.

In other embodiments of the system, the facilitated transport membrane comprises a carrier agent, for example the carrier agent may be a group 11 metal ion.

In more embodiment, the facilitated transport membrane is a hollow-fiber membrane or a spiral-wound flat sheet membrane. In other embodiments, wherein the facilitated transport membrane is humidified. In still more embodiments, the facilitated transport membrane is associated with a module that combines continuous addition of water vapor to form a humidified input stream and selective permeation of an olefin in the humidified input stream using the facilitated transport membrane.

In still more embodiments, the facilitated transport membrane is configured to produce a concentrated paraffin stream or a concentrated olefin stream when producing the enriched feedstock in (b)(i), or the facilitated transport membrane is configured to produce a concentrated paraffin stream, a concentrated olefin stream, and/or a recycle stream that is returned to the distillation column when performing the separation in (b)(ii), or the facilitated transport membrane is configured to produce a concentrated paraffin stream, a concentrated olefin stream, and/or a recycle stream that is returned to the distillation column when performing the separation in (b)(iii) or the facilitated transport membrane is configured to produce a concentrated paraffin stream, a concentrated olefin stream, and/or a recycle stream that is returned to the distillation column when performing the separation in (b)(iv).

In yet other embodiments, (i) a first recycle stream enriched in olefin compared to the side stream is configured to be returned to the distillation column at a location above the first separation tray and (ii) a second recycle stream enriched in paraffin relative to the side stream is configured to be returned to the distillation column at a location below the first separation tray.

In a different aspect, the present disclosure provides a method for producing a polyolefin, the method comprising:
e. enriching an olefin from a feedstock comprising an olefin and a paraffin to produce a reactor feed stream comprising the enriched olefin;
f. in a reactor, polymerizing the enriched olefin in the reactor feed stream to produce a polyolefin and a reactor purge stream, wherein the reactor purge stream comprises paraffin and un-reacted olefin;
g. separating the un-reacted olefin from the reactor purge stream using a module comprising a facilitated transport membrane to produce a recycle stream; and
h. recycling the recycle stream to the reactor.

In some embodiments of the foregoing method, the olefin comprises ethylene, propylene, butylene, butene, or any combination thereof and/or the paraffin comprises ethane, propane, butane, or isobutane.

In different aspects, the feedstock is an output of a thermal cracking process that utilizes naphtha or a heavy liquid stream from a crude distillation unit, for example in some specific aspects the thermal cracking process is a steam cracking or fluidic catalytic cracking (FCC) process.

In more embodiments, the olefin is enriched in step (a) using a distillation column.

In some other exemplary embodiments, the reactor feed stream comprises between about 95 weight percent (wt%) and about 99.5 wt% olefin.

In more embodiments, the method further comprises removing water and sulfurous compounds such as H₂S and COS from the feedstock and/or from the reactor feed stream prior to polymerizing the enriched olefin.

In other aspects of the method, the enriched olefin is polymerized in the presence of H₂, N₂ and a catalyst. In more aspects the reactor further produces a recycle loop that circulates, mixes, and removes heat from the polymerization reaction mixture.

In still further embodiments, the reactor purge stream comprises between about 10 wt% and about 20 wt% paraffin, while different embodiments comprise removing light components prior to separating the un-reacted olefin from the reactor purge stream, for example the light components can be flared or used for fuel.

In more embodiments, the facilitated transport membrane comprises an ionomer, such as a fluorinated ionomer.

In more embodiments, the facilitated transport membrane comprises a carrier agent, such as a group 11 metal ion.

In other further embodiments, the facilitated transport membrane is a hollow-fiber membrane or a spiral-wound flat sheet membrane. In more embodiments, the facilitated transport membrane is humidified.

In still different embodiments, the module combines continuous addition of water vapor to the reactor purge stream to form a humidified reactor purge stream and selective permeation of an olefin in the humidified reactor purge stream using the facilitated transport membrane.

In even more aspects, the present disclosure is directed to a system for producing a polyolefin, the system comprising:
i. a distillation column configured to enrich an olefin from a feedstock comprising an olefin and a paraffin to produce a reactor feed stream comprising the enriched olefin;
j. a reactor configured to polymerize the enriched olefin to produce a polyolefin and a reactor purge stream, wherein the reactor purge stream comprises paraffin and un-reacted olefin; and
k. a membrane separation module comprising a facilitated transport membrane configured to separate the un-reacted olefin from the reactor purge stream to produce a recycle stream, which recycle stream is recycled to the reactor.

In some embodiments of the foregoing system, the olefin comprises ethylene, propylene, butylene, butene, or any combination thereof and/or the paraffin comprises ethane, propane, butane, or isobutane.

In different embodiments, the feedstock is an output of a thermal cracking process that utilizes naphtha or a heavy liquid stream from a crude distillation unit, for example the thermal cracking process may be a steam cracking or fluidic catalytic cracking (FCC) process.

In other different embodiments of the system, the reactor feed stream comprises between about 95 weight percent (wt%) and about 99.5 wt% olefin. In more embodiments, the system further comprises a module configured to remove water and sulfurous compounds such as H₂S and COS from the feedstock and/or from the reactor feed stream prior to polymerizing the enriched olefin.

In other embodiments, the enriched olefin is polymerized in the presence of H₂, N₂ and a catalyst. In certain embodiments, the system further comprises a recycle loop that circulates, mixes, and removes heat from the polymerization reaction mixture, while in other embodiments the reactor purge stream comprises between about 10 wt% and about 20 wt% paraffin.

In more embodiments, the system further comprises a module configured to remove light components prior to separating the un-reacted olefin from the reactor purge stream, for example in some specific embodiments the light components are flared or used for fuel.

In more embodiments, the facilitated transport membrane comprises an ionomer, such as a fluorinated ionomer

In more exemplary embodiments of the system, the facilitated transport membrane comprises a carrier agent, which may be a group 11 metal ion.

In more embodiments, the facilitated transport membrane is a hollow-fiber membrane or a spiral wound flat sheet membrane and/or the facilitated transport membrane is humidified.

In still other embodiments, the module combines continuous addition of water vapor to the reactor purge stream to form a humidified reactor purge stream and selective permeation of an olefin in the humidified reactor purge stream using the facilitated transport membrane.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically illustrates an example of use of a facilitated transport membrane to separate a feed stream, according to the systems and methods described herein.

FIG. 2 schematically illustrates an example of use of a facilitated transport membrane to separate a top product and return a stream to a distillation column, according to the systems and methods described herein.

FIG. 3 schematically illustrates an example of use of a facilitated transport membrane to separate a top product, according to the systems and methods described herein.

FIG. 4 schematically illustrates an example of use of a facilitated transport membrane to separate a bottom product and return a stream to a distillation column, according to the systems and methods described herein.

FIG. 5 schematically illustrates an example of use of a facilitated transport membrane to separate a bottom product, according to the systems and methods described herein.

FIG. 6 schematically illustrates an example of use of a facilitated transport membrane to separate a side stream and return two streams to a distillation column, according to the systems and methods described herein.

FIG. 7 schematically illustrates an example of the systems and methods described herein for use with a splitter column having a heat pump reboiler.

FIG. 8 schematically illustrates an example of the systems and methods described herein for use with a splitter column having a steam reboiler.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” may apply to each of the numerical values in that series of numerical values. For example, greater than or equal to 1, 2, or 3 may be equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” may apply to each of the numerical values in that series of numerical values. For example, less than or equal to 3, 2, or 1 may be equivalent to less than or equal to 3, less than or equal to 2, or less than or equal to 1.

The term “at least one of A and B” and “at least one of A or B” may be understood to mean only A, only B, or both A and B. The term “A and/or B” may be understood to mean only A, only B, or both A and B.

The term “about” as used herein, generally refers to a quantity that is within twenty percent (20%) of the stated quantity.

### Production of Olefins

The systems and methods described herein can be used with various processes for producing olefins.

The production of olefins for the polymerization to polyolefins can be accomplished through the thermal cracking of straight chain paraffinic hydrocarbons by either of two main industrial process, steam cracking and fluidic catalytic cracking (FCC).

The steam cracking process thermally cracks a hydrocarbon stream mixed with steam at high temperature to produce a mixture of a heavier liquid component and light hydrocarbons in the one carbon (C1) to four carbon (C4) boiling range. The light components can be separated in the product recovery section of the cracker by boiling range into fractions, then further separated into olefins and paraffins.

Historically, the cracker feed was a liquid naphtha with a gasoline boiling range. The cracking of this liquid feed produced about 30 wt% ethylene, 13% propylene, and 13% mixed butenes with a balance of H₂, light paraffins, cyclic molecules and heavier components. However, the industry has shifted away from naphtha to liquified petroleum gas (LPG) streams, primarily ethane, which has resulted in an increased yield of ethylene up to 80% but a net reduction in the associated olefin (propylene (2%) and mixed butene's (3%)).

The FCC process can produce olefins by the reaction of heavy liquid streams from the crude distillation unit (gas oil, atmospheric tower bottoms, vacuum tower bottoms) and a catalyst at high temperature to thermally crack the long chain paraffinic molecules into short chain molecules in the one carbon (C1) to six carbon (C6) boiling range, containing olefins, paraffins and cyclic molecules. The heavier products from the reaction can be further refined to produce motor fuels (gasoline, diesel). The lighter LPG components can be separated by boiling range and then as olefins and paraffins. This FCC process typically yields about 8 to 12% LPGs comprising C1 to C4 with the proportion of olefins to paraffins highly dependent on feed stock and operating conditions.

As the steam cracker has shifted towards a more economical feed (ethane) and the production of LPGs from the FCC process is reduced as the global demand for motor fuels declines, the production of the associated olefins from these processes is also reduced. The reduction in the olefin production capacity contrasts with the worldwide demand for plastics that is growing at 3.5 to 4.5 compound annual growth rate (CAGR).

### Enrichment of Olefins

Olefins can be enriched using a distillation column. For separation of C2's (i.e., ethylene from ethane), the distillation column can be a cryogenic column. The top product can be condensed and the reboiler is heated by a propylene refrigeration system. The reboiler may operate at approximately 450 psig and 10 °F. Marketable C2 products can include polymer grade ethylene (PGE), which can include at least 99.95 mol% ethylene.

There are two types of distillation columns in use for C3 separations, steam re-boiled distillation and heat pump distillation. The two distillation designs can provide equivalent separations, but the heat pumped distillation can be more energy efficient and is typically preferred when waste heat is not available for the steam re-boiled design. The steam re-boiled column typically operates with a pressure of 230 psig and a temperature of 120 °F at the reboiler. Due to the low temperature at the reboiler, very low-pressure steam or possibly condensate can be used for heating. The heat pumped distillation operates at about 165 psig and 100 °F at the reboiler, but no external heat source is required. Rather, the overhead vapor is compressed and the heat of compression is used to heat the reboiler. Feed to the C3 splitter columns from an upstream catalytic cracker may be refinery grade propylene (RGP), which contains 65% to 85% propylene with the balance being primarily propane, or if the upstream process is propane dehydrogenation, the feed to the C3 splitter will be 50% propylene and the balance propane. In either case, columns are typically operated to produce two marketable product streams. These marketable streams can include: HD5 propane (containing less than 5% propylene by liquid volume); Chemical grade propylene (CGP) (containing less than 8 wt% propane); and Polymer grade propylene (PGP) (containing at least 99.5 wt% propylene).

For enrichment of C4 olefin, the C4 fraction from a catalytic cracker is a mixture of mono-olefin isomers (1-butene, cis-2-butene, trans-2-butene, and isobutylene), di-olefin isomers (1,3-butadiene and 1,2 butadiene), and paraffin isomers (n-butane and isobutane). The relative volatilities of the C4 fraction components are very close, making conventional distillation infeasible. Instead, a complex and costly series of separation processes must be used. The 1,3 butadiene is usually the first component separated out by extractive distillation using a polar solvent. The 1,2 butadiene is then hydrogenated to produce a stream containing only mono-olefins and paraffins. The 2-butene can then be readily separated out by distillation but the paraffins will distribute between the top and bottom products. Depending on the purity required, the paraffins may be separated from 2-butene by extractive distillation. The 1-butene rich product stream will also contain isobutene and paraffins. The isobutene can be separated by sulfuric acid extraction. Finally, extractive distillation is used to separate 1-butene from the paraffins. Other variations of the process are possible.

The systems and methods described herein can be used with any of the aforementioned C2, C3, or C4 separation processes.

### Systems and Methods for Increasing Capacity of Olefin-Paraffin Splitter Columns

The membrane separation process described herein can be an alternative to replacing an existing distillation column or adding an additional distillation column. The membrane separation process may be retrofitted to an existing column in order to separate a stream associated with the column for the purpose of increasing the column's capacity.

The membrane process can be used to separate any of the streams associated with the distillation (splitter) columns including the feed, top product, bottom product, or side stream.

The membrane process can separate all or only part of any such available stream. The membrane process may comprise a single membrane stage or multiple stages or steps.

The membrane process may produce two product streams that are withdrawn from the separation process or it may produce one product stream (e.g., with the second stream leaving the membrane process being returned to the column). If the membrane process separates a side stream, one, two, or more streams leaving the membrane process may be returned to the column. In most cases, feed to the membrane is in the vapor state.

In an aspect, described herein is a method for separating olefin from paraffin. The method can include enriching an olefin from a feedstock comprising an olefin and a paraffin using a distillation column, where the distillation column (i) has a plurality of separation trays, (ii) produces a top product that is enriched in olefin relative to the feedstock, and (iii) produces a bottom product that is depleted in olefin relative to the feedstock.

The methods can further include using a facilitated transport membrane that is capable of separating olefin from paraffin, performing one or more of: (i) separating olefin from paraffin in the feedstock to produce an enriched feedstock, which enriched feedstock is enriched in olefin relative to the feedstock, and wherein the enriched feedstock is fed to the distillation column (i.e., separation of column feed), (ii) separating olefin from paraffin in the top product of the distillation column, (iii) separating olefin from paraffin in the bottom product of the distillation column, and (iv) withdrawing a side stream from the distillation column in proximity to a first separation tray of the plurality of separation trays and separating olefin from paraffin in the side stream. In some embodiments, at least two of (i-iv) are performed. In some embodiments, at least three of (i-iv) are performed. In some embodiments, all of (i-iv) are performed.

When applying the systems and methods described herein to a C2 splitter, streams returned to the column must be cooled to cryogenic temperatures, while feed to the membrane must be in vapor state and heated to at least 4 0°F to avoid the risk of freezing the humidification water. Therefore, heat transfer can be integrated into the systems and methods (e.g., using one or more heat exchangers).

### Membrane Separation of Column Feed

The systems and methods can include using a facilitated transport membrane that is capable of separating olefin from paraffin to separate olefin from paraffin in the feedstock to produce an enriched feedstock, which enriched feedstock is enriched in olefin relative to the feedstock, and wherein the enriched feedstock is fed to the distillation column (i.e., separation of column feed).

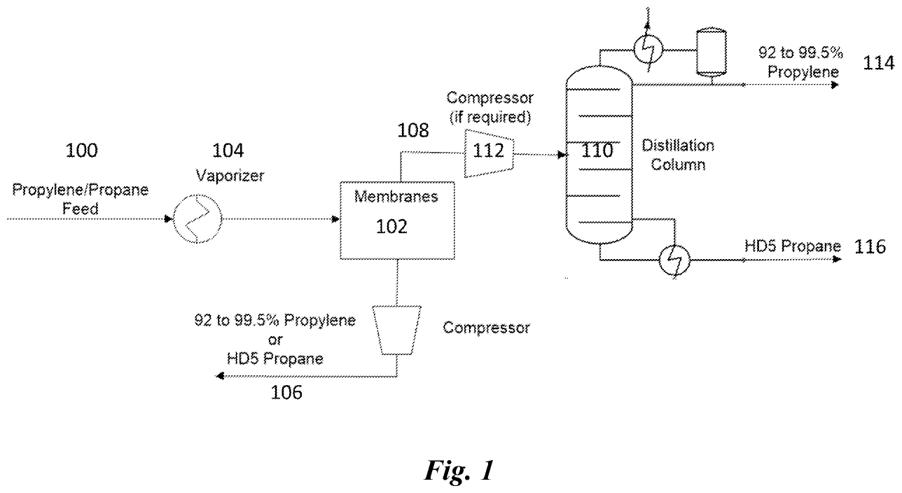

In the process configuration shown in FIG. 1, the membrane module separates the feed to the column. This configuration may be applied to either a C2 or C3 splitter. All of the feed or only a portion of it may be separated by the membrane process. If only a portion of the feed is separated by the membrane, the membrane process may produce two separate product streams or produce two streams that are returned to the column (not shown). In the embodiment shown in FIG. 1, the choice of product (olefin rich or paraffin rich) produced by the membrane process and whether it is single stage or multi-stage/multi-step can depend on the feed composition. The likeliest configuration is a single stage membrane producing an olefin rich product. Compressors can use aftercoolers and intercoolers if the compressor has more than one compression stage Additional equipment that is not shown in the figures can be used for humidifying the membranes and drying the product streams.

Continuing with FIG. 1, a feedstock 100 can be fed to the membrane module 102, e.g., after passing through a vaporizer 104. A first stream 106 can be produced by the membrane module, e.g., that can be either an olefin or paraffin stream, optionally having specifications that make the first stream commercially marketable. A second stream 108 (referred to herein as an enriched feedstock) can be fed to the distillation column 110, optionally through a compressor 112. From the enriched feedstock 108, the distillation column 110 can produce a top product 114 (e.g., olefin) and a bottom product 116 (e.g., paraffin).

Using facilitated transport membranes to “pre-process” the feed can increase the overall capacity of the separation process without retrofitting or replacing the distillation column.

### Membrane Separation of Column Top Product

The systems and methods can include using a facilitated transport membrane that is capable of separating olefin from paraffin to separate olefin from paraffin in the top product of the distillation column.

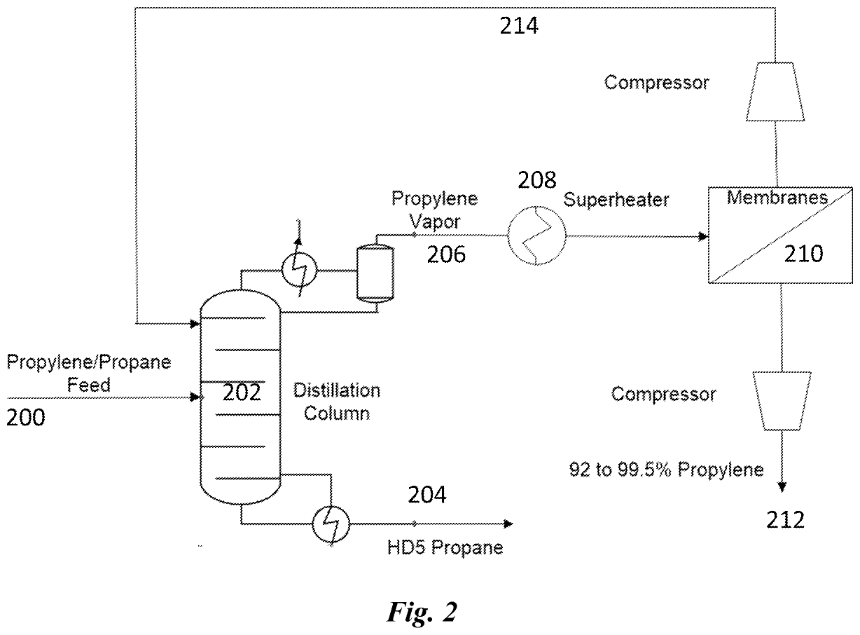

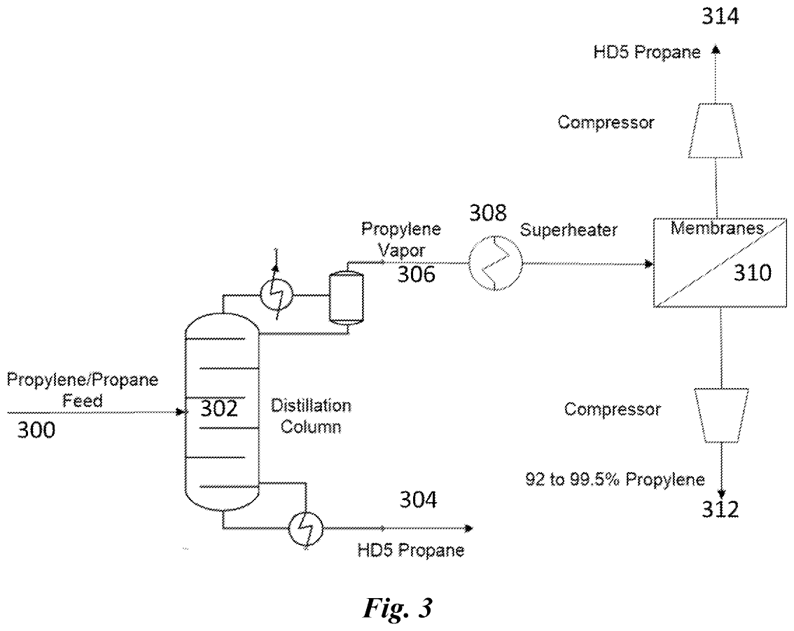

In the process configuration shown in FIG. 2 and FIG. 3, the membrane module can complete the separation of the distillate product to meet purity specifications. This configuration can be applied to a C3 splitter. The process in FIG. 2 will typically use a single stage membrane process, but a multi-stage/multi-step process is possible. In the embodiment in FIG. 3, the membrane module produces two product streams rather than returning one stream to the column. This embodiment will likely use a multi-stage/multi-step membrane process. In both cases, the compressors can use aftercoolers and intercoolers if the compressor has more than one compression stage. Additional equipment that is not shown in the figures can be used for humidifying the membranes and drying the product streams.

Turning to FIG. 2, a feedstock 200 can be fed to a distillation column 202 to produce a bottom product 204 and a top product 206. The top product 206 can be superheated 208 and fed to the membrane separation module 210. One product of the membrane separation can be an enriched olefin stream 212, while another stream can be returned 214 to the splitter 202.

Turning to FIG. 3, a feedstock 300 can be fed to a distillation column 302 to produce a bottom product 304 and a top product 306. The top product 306 can be superheated 308 and fed to the membrane separation module 310. One product of the membrane separation can be an enriched olefin stream 312, while another stream can be an enriched paraffin stream 314.

### Membrane Separation of Column Bottom Product

The systems and methods can include using a facilitated transport membrane that is capable of separating olefin from paraffin to separate olefin from paraffin in the bottom product of the distillation column.

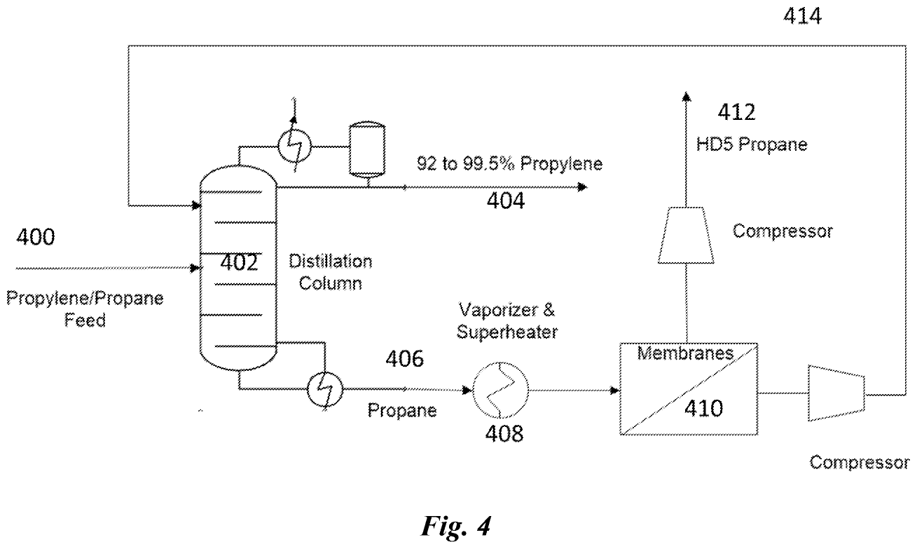

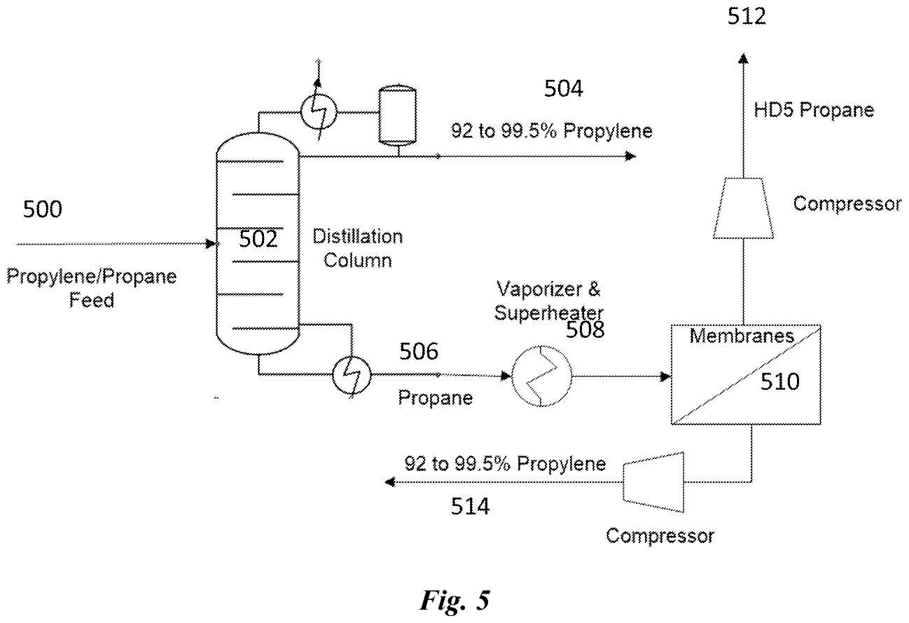

In the process configurations shown in FIG. 4 and FIG. 5, the membrane process completes the separation of the bottom product to meet purity specifications. These configurations may be applied to either a C2 or C3 splitter. The embodiment in FIG. 4 can use a single stage membrane process, but a multi-stage/multi-step process is possible. In the embodiment of FIG. 5, the membrane process produces two product streams rather than returning one stream to the column. This case can use a multi-stage/multi-step membrane process. In both instances, the compressors can use aftercoolers and intercoolers if the compressor has more than one compression stage. Additional equipment that is not shown in the figures can be used for humidifying the membranes and drying the product streams.

Turning to FIG. 4, the feedstock 400 can be fed to a distillation column 402 to produce a top product 404 and a bottom product 406. The bottom product can be vaporized and/or superheated 408 and fed to a membrane separation module 410. One product of the membrane separation can be an enriched paraffin stream 412, while another stream can be an enriched olefin stream 414 that is fed back to the splitter 402.

Turning to FIG. 5, the feedstock 500 can be fed to a distillation column 502 to produce a top product 504 and a bottom product 506. The bottom product can be vaporized and/or superheated 508 and fed to a membrane separation module 510. One product of the membrane separation can be an enriched paraffin stream 512, while another stream can be an enriched olefin stream 514.

### Membrane Separation of Column Side Stream

The systems and methods can include using a facilitated transport membrane that is capable of separating olefin from paraffin to withdrawing a side stream from the distillation column in proximity to a first separation tray of the plurality of separation trays and separate olefin from paraffin in the side stream.

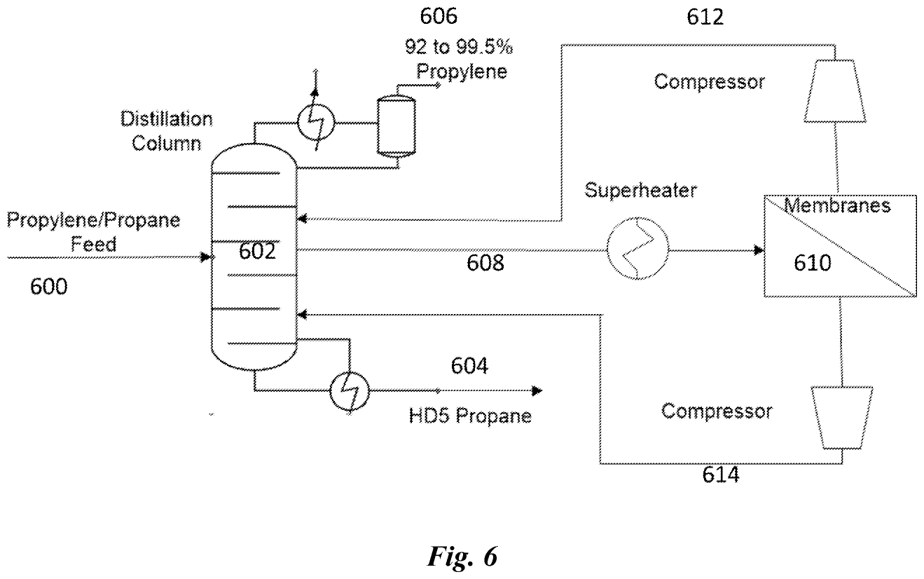

In the embodiment shown in FIG. 6, the membrane process separates a side stream from the column. This configuration may be applied to either a C2 or C3 splitter. The membrane process may return zero, one, two streams to the column. FIG. 6 illustrates the case of two streams being returned to the column. The choice of which if any streams are returned to the column and whether the membrane process is single stage or multi-stage/multi-step can depend at least in part on the side stream composition. As in the other configurations described herein, compressors can use aftercoolers and intercoolers if the compressor has more than one compression stage. Additional equipment that is not shown can be used for humidifying the membranes and drying the product streams.

Continuing with FIG. 6, the feedstock 600 can be fed to the distillation column 602 to be separated into a top product 604 and a bottom product 606. A side stream 608 can be withdrawn from the column 602 and taken to a membrane module 610 to be split into a fist stream 612 and a second stream 614. Any number of the streams coming from the membrane module 610 can be returned to the distillation column 602.

### Systems and Methods for Olefin Recovery and Recycling

The membrane separation process described herein may be retrofitted to an existing polyolefin polymerization reactor purge stream (or incorporated into a newly constructed polyolefin process) to separate the olefin and return it to the reactor feed stream, while simultaneously yielding a paraffin rich stream that may be sold or returned to the cracker process for further processing.

In an aspect, provided herein is a method for producing a polyolefin. The method can comprise enriching an olefin from a feedstock comprising an olefin and a paraffin to produce a reactor feed stream comprising the enriched olefin. The method can further comprise, in a reactor, polymerizing the enriched olefin in the reactor feed stream to produce a polyolefin and a reactor purge stream, wherein the reactor purge stream comprises paraffin and un-reacted olefin. The method can further comprise separating the un-reacted olefin from the reactor purge stream using a module comprising a facilitated transport membrane to produce a recycle stream and recycling the recycle stream to the reactor.

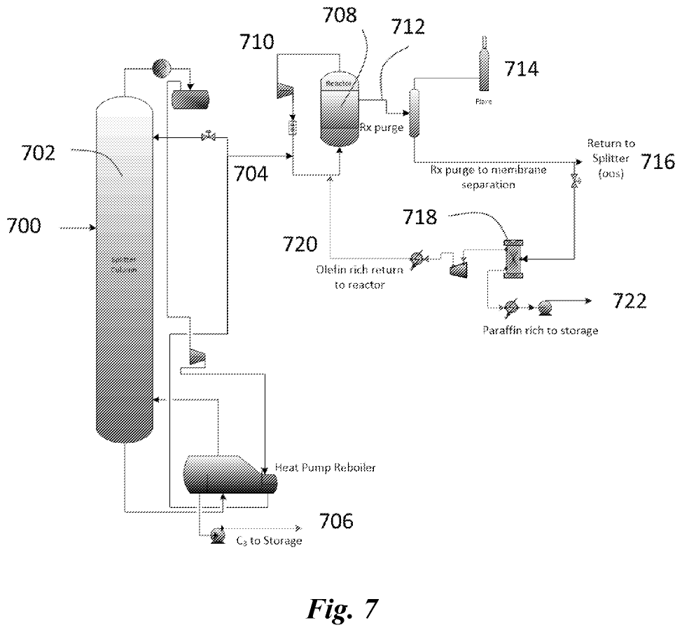

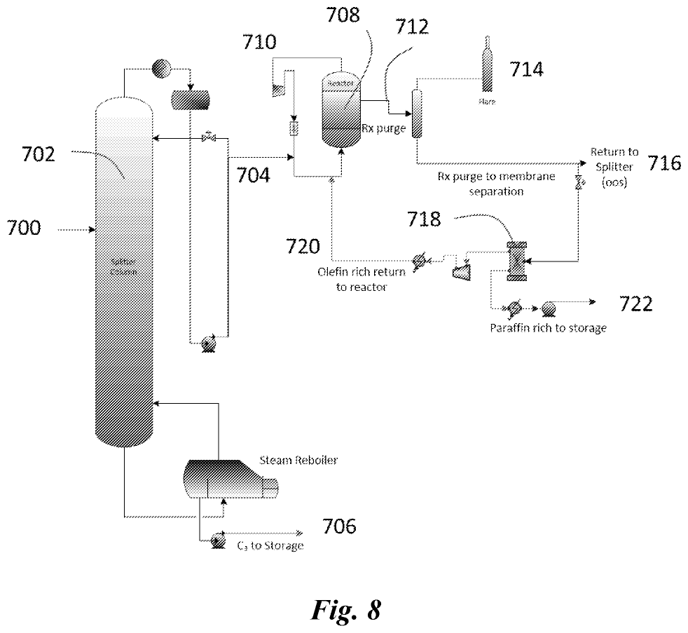

FIG. 7 schematically illustrates an example of the systems and methods described herein for use with a splitter column having a heat pump reboiler. Similarly, FIG. 8 schematically illustrates an example of the systems and methods described herein for use with a splitter column having a steam reboiler. Referring to commonly indicated modules and units therein, a feedstock 700 can be sent to a distillation column 702 to produce an overhead (light) stream 704 comprising predominantly olefin and a bottoms (heavy) stream 706 comprising predominantly paraffin. In some cases, the feedstock is an output of a thermal cracking process that utilizes naphtha or a heavy liquid stream from a crude distillation unit. In some embodiments, the thermal cracking process is a steam cracking or fluidic catalytic cracking (FCC) process. In some embodiments, the olefin comprises ethylene, propylene, butylene, butene, or any combination thereof. In some cases, the paraffin comprises ethane, propane, butane, or isobutane

Continuing with FIG. 7 and FIG. 8, the overhead stream can be used directly, or with additional processing, as a reactor feed stream comprising the enriched olefin. Additional processing can include removing water and sulfurous compounds such as H₂S and COS from the feedstock and/or from the reactor feed stream prior to polymerizing the enriched olefin (not shown). The reactor feed stream can be sent directly into the reactor 708. In some cases, as shown here, the reactor feed stream can be combined with a reactor recycle loop 710 that circulates, mixes, and removes heat from the polymerization reaction mixture. In some embodiments, the reactor feed stream comprises between about 95 weight percent (wt%) and about 99.5 wt% olefin. The enriched olefin can be polymerized in a reactor 708 to produce a polyolefin and a reactor purge stream 712. The enriched olefin can be polymerized in the presence of H₂, N₂ and a catalyst.

The reactor purge stream 712 can include between about 10 wt% and about 20 wt% paraffin, with the balance the more valuable olefins. In some cases, the light components are removed prior to separating the un-reacted olefin from the reactor purge stream. The light components 714 can be flared or used for fuel. Any portion of the reactor purge stream 716 can be returned to the distillation column 702. However, returning the purge stream to the distillation column can result in reduced capacity for separation of fresh feedstock 700 and increase energy demand of the column 702. Therefore, some (including, e.g., substantially all) of the reactor purge stream can be sent to the membrane separation module 718 as described herein.

The membrane separation 718 (i.e., comprising a facilitated transport membrane) can result in an olefin-rich recycle stream 720 that can be returned to the polymerization reactor 708. Additionally, a paraffin-rich stream 722 can be a marketable product or cracked to produce additional olefin.

The membrane process may separate all or only part of an available stream. In some cases, the membrane separates at least about 10%, at least about 20%, at least about 30%, at least about 40%, at least about 50%, at least about 60%, at least about 70%, at least about 80%, or at least about 90% of the purge stream. In some instances, the membrane separates at most about 10%, at most about 20%, at most about 30%, at most about 40%, at most about 50%, at most about 60%, at most about 70%, at most about 80%, or at most about 90% of the purge stream.

The process can use a multi-stage/multi-step membrane process. The membrane process may consist of a single membrane stage or multiple stages (e.g., 2, 3, 4, 5 stages) or multiple steps (e.g., 2, 3, 4, 5 steps). The membrane process may produce two product streams that are withdrawn from the separation process. In some cases, produces one product stream (i.e., the second stream leaving the membrane process being returned to the distillation column). The feed to the membrane can be in the vapor state.

In some cases, the compressors can use aftercoolers and intercoolers if the compressor has more than one compression stage. Additional equipment that is not shown in the figures can be used for humidifying the membranes and drying the product streams.

Overall, the systems and methods described herein can increase overall plant polyolefin production by at least about 2%, at least about 3%, at least about 4%, at least about 5%, at least about 6%, at least about 7%, at least about 8%, at least about 9%, at least about 10% relative to simply returning the purge stream to the distillation column. In some instances, the plant production is increased by between about 2.5% to 8%.

The systems and methods can also increase production of a paraffin rich product.

Marketable C 2 products can include chemical grade ethylene (CGE) having greater than 95 mol% ethylene, or a concentrated ethane stream having greater than 95 mol% ethane. Marketable C3 products can include HD5 propane (containing less than 5% propylene by liquid volume), chemical grade propylene (CGP) having less than 8 wt% propane, or polymer grade propylene (PGP) having at least about 99.5 wt% propylene.

### Polyolefin Production Processes

The systems and methods described herein can be used with various processes for producing polyolefins. There are a several technologies for converting olefins to polyolefins. For polypropylene the four primary technologies include the Spheripol™ liquid phase process (of Lyondell Bassell), the Unipol™ gas phase process (of W.R. Grace), the Novolen™ gas phase process, and the Innovene™ gas phase process (of Ineos).

In these process, the feed from the overhead of the splitter column to C3 polymerization reactors can range from 95% to 99.5+ wt% olefin, and the C2 polymerization reactors ranges from 98% to 99.95% olefin, with the balance being paraffin. The preparation of the feed for the reaction process includes the removal of water and sulfuric containments (H₂S, COS). After drying and pretreatment, the feed can be sent to the polyolefin reactor where it is mixed with H₂ and N₂ and a catalyst to promote the polymerization reaction. The polymerization reactor gas phase recycle loop circulates the reaction mixture, removing heat from the process and providing mixing and agitation of the reaction products. As the feed contains some concentration of paraffinic components that do not participate in the reaction, the paraffin concentrates within the reactor, slowing the reaction rate. To improve kinetics, the concentrated paraffin is continually withdrawn from the reactor, resulting in the removal of both the concentrated paraffins (10-20%) with the balance containing the valuable olefins. The purge stream can be sent through a recovery system where light ends are removed to fuel or flare and the heavier hydrocarbons are returned to the splitter columns for reprocessing and return to the reactors.

Traditionally, there were three process options for the reactor purge stream. First, one could recycle the material back through the splitter column. Alternatively, one could send the stream to fuel or burn the material in a flare. While each of these options are viable, each result in additional cost to the plant. For example, recycling to the splitter column can increases energy demand and limit fresh feed to column. Sending to fuel can realize only the heating value of olefin instead of conversion to polyolefin. While flaring results in complete loss of valuable feedstock and increased greenhouse gas emissions.

In contrast, the systems and methods described herein provide a more attractive option to the polyolefin plant. The systems and methods developed herein can use a facilitated transport membrane with reactor purge stream to realize numerous benefits.

### Facilitated Transport Membranes

The systems and methods described herein can use facilitated transport membranes.

The facilitated-transport membrane may incorporate a carrier agent to increase the solubility of certain components in the gaseous feed stream through reversible reaction or complexation mechanisms and thereby preferably “facilitate” their transport through the membrane. The carrier agents may be covalently or electrostatically bound within the membrane to prevent their migration or loss from the membrane during use. Facilitated-transport membranes that are fabricated from polymer materials that are ionomers are highly useful in the separations described herein. A carrier agent such as silver ions for separation of olefins may be electrostatically bound within the ionomer.

The ionomer can be fluorinated. The facilitated transport membrane can also include a carrier agent such as a group 11 metal ion (e.g., copper, silver, gold). In cases, the facilitated transport membrane is a hollow-fiber membrane.

Ionomers can be used for the gas-separation layer of the thin-film composite membranes. As used herein, an ionomer is a copolymer that contains covalently-bound ionic-pendant groups such as sulfonic acid, sulfonate, carboxylic acid, carboxylate, phosphate, phosphonium, or ammonium. As used herein, the ionomer equivalent weight is the weight of ionomer containing one mole of sulfonate group. The ionomer equivalent weight (EW) can be less than 5000 grams per mole, less than 2000, or between 500 and 800-g/mole.

Ionomers that are copolymers containing sulfonic acid or sulfonate groups can be useful for fabrication of the gas-separation layer. Suitable ionomers and membranes include those described in U.S. Pat. Nos. 5,191,151; 10,639,591; and 10,029,248, each of which are hereby incorporated by reference. Suitable ionomers can comprise repeat units A and B in which A is a polymerized derivative of a fluorinated monomer and B comprises sulfonate groups. The ionomers can contain 50% or more carbon-fluorine groups to carbon-fluorine groups plus carbon-hydrogen groups. Some ionomers are fluoropolymers in which there are no carbon-hydrogen groups in the polymer-backbone repeating units. Examples of the latter ionomers include copolymers comprising polymerized repeat units of tetrafluoroethylene and a perfluorovinyl ether monomer, having a pendant sulfonate group such as for example Nafion® (Chemours, Wilmington Del.), and Aquivion® (Solvay, Houston Tex.).

The gas-separation layer can be fabricated from an ionomer solution that is substantially free of other dissolved ionic species not associated with the ionomer. Other ionic (cationic or anionic) species not associated with the ionomer can include an ion where the counter ion is not covalently bound to the ionomer. A substantially-free ionomer solution can have concentrations of other cationic species at molar ratios to ionomer sulfonate-groups (—SO₃⁻) that are between 0 and less than 0.37, which is also less than the equivalent 0.5:1 ratio of g-equivalents of silver (from the added silver compound) to g-equivalents of —SO₃⁻ groups. The substantially-free ionomer solution may comprise more than one type of ionomer, or associated counter ions. For example, an ionomer solution comprising covalently bound sulfonate groups may have associated counter ions that are different from silver such as for example H⁺, Na⁺, K⁺, ammonium, alkyl ammonium, or mixtures therefrom. The different counter ions may be exchanged for silver to activate the thin-film composite membrane after fabrication.

The gas-separation layer thickness can have a significant influence on the membrane cost and productivity of the separation process per unit area. The gas-separation layer can be thin (0.01-μm to 5-μm). The gas-separation layer thickness can be optimized such that both the olefin permeance through the thin-film composite membrane and selectivity over other gases is high per unit area.

The porous-layer support may be in the form of a flat sheet, hollow fiber, or tube. The porous-layer support reinforces the thin gas-separation layer and helps to further mechanically strengthen the thin-film composite as a whole such that the membrane may be fabricated into more complex geometries such as spiral-wound or hollow-fiber membrane modules. The porous-layer support may also comprise an even stronger backing material such as porous non-woven polyester or polypropylene. Suitable porous-layer support materials include but are not limited to polyvinylidine fluoride, expanded polytetrafluoroethylene, polyacrylonitrile, polysulfone, polyether ether ketone (PEEK), and polyethersulfone. Porous inorganic substrates such as porous silica or alumina are also suitable support materials. Permeate gases should flow relatively unobstructed through the usually much thicker porous-layer support having a preferred porosity that is 40% or greater. The porous-layer support average pore size is preferably less 0.1-μm and more preferably between 0.01 and 0.03-μm.

The gas-separation layer in the thin-film composite membrane is coplanar and in direct contact with the porous-layer support. The gas-separation layer may also be predominantly laminar. By “predominantly laminar” is meant that the surface or interfaces of two or more distinct layers have 50% or more of at least one layer material not interpenetrating the pores of another layer.

The gas-separation layer in the thin-film composite membrane may be subjected to a thermal treatment step “annealed” to further improve mechanical durability, long-term olefin permeance and selectivity, and resistance to degradation from contact with liquid water. The ionomer in the gas-separation layer can be annealed by heating the thin-film composite membrane to near or above the glass transition temperature of the ionomer. The exact glass transition temperature will be dependent on the ionomer composition and the associated counter ion. Generally, annealing temperatures are between 50 and 200 °C.

The gas-separation layer comprising sulfonic-acid, or sulfonate-salt groups other than silver sulfonate, is initially relatively inactive. The thin-film composite membrane can be activated by exchange of protons or other cations for silver in the gas-separation layer. For example, the exchange may be carried out by contacting the exposed gas-separation layer surface with a solution comprising water and a soluble and ionizable silver compound such as silver nitrate. A sufficient level of exchange can quickly occur for a thin gas-separation layer as evidenced by the observed high permeance (>100-GPU) and selectivity (>25) for propylene over propane after less than 1 minute of contact with aqueous silver nitrate at ambient (20 to 25 °C) temperature.

The thin-film composite membrane can be highly useful for the separation of olefins from paraffins and for separation of olefins from other non-hydrocarbon gases such as helium, hydrogen, nitrogen, or argon. The membrane feed-side can be exposed to a flowing gaseous composition comprising an olefin. A driving force can be provided in which the olefin pressure on the membrane feed-side is higher than on the membrane permeate side. Separation of the olefin in the gaseous composition occurs through the membrane producing a composition at the membrane permeate-side having a higher concentration of olefin than the membrane feed-side.

Separation may also be enhanced by having water vapor in the composition and/or by using a sweep gas on the membrane permeate-side, which functions to reduce the permeate concentration. For example, a sweep gas may comprise an inert gas such as water vapor or nitrogen.

### Humidification

The facilitated transport membrane used herein can be humidified. The humidification can be provided by hydrating the permeate side of a vessel that contains the facilitated transport membrane, as described in U.S. patent application Ser. No. 17/276,639, which is incorporated herein by reference. In some embodiments, the module combines continuous addition of water vapor to the reactor purge stream to form a humidified reactor purge stream and selective permeation of an olefin in the humidified reactor purge stream using the facilitated transport membrane as described in U.S. patent application Ser. No. 17/772,247, which is incorporated herein by reference.

A suitable method for humidification of a facilitated-transport membrane includes a method in which a non-selective hydration fluid comprising liquid water is brought within the permeate side of a pressure vessel that contains the facilitated-transport membrane (permeate-side humidification). This is unlike humidification of a permeate-gas sweep in which a gas or gas composition that is different than the permeate gas is separately humidified and subsequently passed through the permeate side of the pressure vessel. Here, the non-selective hydration fluid comprising liquid water and the permeate-side interface of the facilitated-transport membrane are in communication within the pressure vessel (i.e., liquid water or water vapor from the non-selective hydration fluid is contacting the permeate-side interface of the facilitated-transport membrane). The facilitated-transport membrane may be in the form of a flat sheet, hollow fiber, or spiral-wound membrane module. The facilitated-transport membrane can be non-porous and may also comprise other layers such as a high-diffusion rate (gutter) layer and a porous support in a composite membrane construction.

Permeate-side humidification at high operating feed-pressures can be less complex than traditional feed-gas humidification. That is, requirements for precise temperature control between a separate humidification unit-operation of a large feed-gas flow and the membrane may be minimized with use of the non-selective hydration-fluid comprising liquid water within the permeate-side of the pressure vessel. The non-selective hydration fluid may also be at an equivalent or slightly lower pressure than the permeate gas and may also function as a permeate sweep to reduce the permeate concentration at the permeate-side interface and enhance overall membrane selectivity. The permeate gases form bubbles within the non-selective hydration fluid that either move away from the permeate-side interface due to buoyancy or are swept away in a flowing or recirculating non-selective hydration-fluid system. The non-selective hydration fluid may be replenished as it diffuses into the membrane, evaporates, or moves away from the membrane with the permeate bubbles.

The systems and methods described herein can use a module for separation of a gaseous feed stream that combines concurrent humidification and selective permeation within the same unit of operation. The humidification and selective permeation module can comprise two sets of hollow fibers; humidification hollow fibers containing fluid comprising liquid water within their hollow cores, and facilitated-transport hollow fibers that comprise a nonporous facilitated-transport membrane. Continuous humidification of the feed stream within the module can be provided by the humidification hollow fibers while selective permeation of components in the feed stream occurs through the facilitated-transport membrane of the facilitated-transport hollow fibers. The humidity level in the gaseous feed stream can be maintained along the flow path of the feed stream and continuously replenished due to the humidification hollow fibers and facilitated-transport hollow fibers that may be closely overlapping, aligned, intermingled, layered, or interlaced with each other. Furthermore, humidification within the selective-permeation module is less complex than traditional feed-gas humidification since requirements for precise temperature control of a gaseous feed stream between separate unit operations can be eliminated or reduced. The humidification hollow fibers can provide a more uniform hydration of the facilitated-transport membrane of the facilitated-transport hollow fibers and result in more consistent permeability and selectivity throughout the length of the module.

The humidification hollow fibers and the facilitated-transport hollow fibers can be porous (e.g., microporous). The hollow fibers may be constructed of the same or different materials. The humidification hollow fibers contain fluid comprising liquid water in their lumen as the source of the humidification. The walls of the humidification hollow fibers permeate water vapor but also function as a barrier preventing liquid water from entering the flow path of the gaseous feed stream and contacting the facilitated-transport membrane, which may be detrimental to overall performance. The facilitated-transport hollow fibers are also permeable and function as a porous support for a nonporous facilitated-transport membrane in a composite construction. The composite construction may include additional layers such as a high-diffusion rate (gutter) layer which can help to reduce interfacial resistance between the facilitated-transport hollow fibers and the nonporous facilitated-transport membrane and help increase overall permeance and selectivity.

The humidification and selective permeation module as described herein may be used where humidification is desirable or required for better gas-separation efficiency using a facilitated-transport membrane, especially when operating at higher stage cuts where a larger fraction of the feed stream permeates the membrane.

Overall, the systems and methods described herein can increase the separation capacity of the column, e.g., by at least to 10%, at least 20%, at least 30%, or at least 40%.

The systems and methods described herein can reduce energy input for processing the increased feed by at least about 40%, at least about 50%, at least about 60%, at least about 70%, at least about 80%, or at least about 90% compared to distillation not using the facilitated transport membranes.

The systems and methods can change the energy mix for separation of the increased feed to be more weighted to electrical energy. When produced from renewables, this can reduce greenhouse gas emissions for processing the increased feed by at least 20%, at least 30%, at least 40%, at least 50%, at least 60%, or at least 70% compared to distillation not using the facilitated transport membranes.

The systems and methods described herein can eliminate or delay the need to replace an existing column or add an additional distillation column, while still gaining capacity by at least 10%, at least 20%, at least 30%, at least 40%, or at least 50%.

This increased capacity can come with a lower capital cost than increasing distillation capacity and can provide an energy cost savings for additional capacity relative to distillation.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure. The present application claims priority to U.S. Provisional Application No. 63/471,058, filed on Jun. 5, 2023, and U.S. Provisional Application No. 63/471,055, filed Jun. 5, 2023, in the United States Patent Office, the entire contents and disclosure of which are incorporated herein by reference.

1. A method for separating olefin from paraffin, the method comprising:
a. enriching an olefin from a feedstock comprising an olefin and a paraffin using a distillation column, wherein the distillation column (i) has a plurality of separation trays, (ii) produces a top product that is enriched in olefin relative to the feedstock, and (iii) produces a bottom product that is depleted in olefin relative to the feedstock; and
b. using a facilitated transport membrane that is capable of separating olefin from paraffin, performing one or more of:
i. separating olefin from paraffin in the feedstock to produce an enriched feedstock, which enriched feedstock is enriched in olefin relative to the feedstock, and wherein the enriched feedstock is fed to the distillation column,
ii. separating olefin from paraffin in the top product of the distillation column,
iii. separating olefin from paraffin in the bottom product of the distillation column, and
iv. withdrawing a side stream from the distillation column in proximity to a first separation tray of the plurality of separation trays and separating olefin from paraffin in the side stream.

2-4. (canceled)

5. The method of claim 1, wherein the olefin comprises ethylene, propylene, butylene, butene, or any combination thereof.

6. The method of claim 1, wherein the paraffin comprises ethane, propane, butane, or isobutane.

7. The method of claim 1, wherein the feedstock is an output of a thermal cracking process that utilizes naphtha or a heavy liquid stream from a crude distillation unit.

8. (canceled)

9. The method of claim 1, further comprising, in a reactor, polymerizing an enriched olefin stream to produce a polyolefin.

10. (canceled)

11. The method of claim 1, wherein the facilitated transport membrane comprises an ionomer.

12. The method of claim 11, wherein the ionomer is fluorinated.

13. The method of claim 11, wherein the facilitated transport membrane comprises a carrier agent.

14. (canceled)

15. The method of claim 1, wherein the facilitated transport membrane is a hollow-fiber membrane or a spiral-wound flat sheet membrane.

16. The method of claim 1, wherein the facilitated transport membrane is humidified.

17. The method of claim 1, wherein the facilitated transport membrane is associated with a module that combines continuous addition of water vapor to form a humidified input stream and selective permeation of an olefin in the humidified input stream using the facilitated transport membrane.

18. The method of claim 1, wherein producing the enriched feedstock in (b)(i), further produces a concentrated paraffin stream or a concentrated olefin stream.

19-22. (canceled)

23. A system for separating olefin from paraffin, the system comprising:
a. a distillation column having a plurality of trays, which distillation column is configured to (i) enrich an olefin from a feedstock comprising an olefin and a paraffin, (ii) produce a top product that is enriched in olefin relative to the feedstock, and (iii) produces a bottom product that is depleted in olefin relative to the feedstock; and
b. facilitated transport membrane that is capable of separating olefin from paraffin and configured to perform one or more of:
i. separate olefin from paraffin in the feedstock to produce an enriched feedstock, which enriched feedstock is enriched in olefin relative to the feedstock, and wherein the enriched feedstock is configured to be fed to the distillation column,
ii. separate olefin from paraffin in the top product of the distillation column,
iii. separate olefin from paraffin in the bottom product of the distillation column, and
iv. withdraw a side stream from the distillation column in proximity to a first separation tray of the plurality of separation trays and separate olefin from paraffin in the side stream.

24-44. (canceled)

45. A method for producing a polyolefin, the method comprising:
a. enriching an olefin from a feedstock comprising an olefin and a paraffin to produce a reactor feed stream comprising the enriched olefin;
b. in a reactor, polymerizing the enriched olefin in the reactor feed stream to produce a polyolefin and a reactor purge stream, wherein the reactor purge stream comprises paraffin and un-reacted olefin;
c. separating the un-reacted olefin from the reactor purge stream using a module comprising a facilitated transport membrane to produce a recycle stream; and
d. recycling the recycle stream to the reactor.

46-64. (canceled)

65. A system for producing a polyolefin, the system comprising:
a. a distillation column configured to enrich an olefin from a feedstock comprising an olefin and a paraffin to produce a reactor feed stream comprising the enriched olefin;
b. a reactor configured to polymerize the enriched olefin to produce a polyolefin and a reactor purge stream, wherein the reactor purge stream comprises paraffin and un-reacted olefin; and
c. a membrane separation module comprising a facilitated transport membrane configured to separate the un-reacted olefin from the reactor purge stream to produce a recycle stream, which recycle stream is recycled to the reactor.

66-83. (canceled)
